(19) United States
(12) Patent Application Publication
(10) Pub. No.: US 2009/0258914 A1
(43) Pub. Date: Oct. 15, 2009

(54) Inhibitor of Anti-Apoptotic Proteins

(75) Inventor: Maurizio Pellecchia, La Jolla, CA (US)

Correspondence Address: DLA PIPER LLP (US), 4365 EXECUTIVE DRIVE, SUITE 1100, SAN DIEGO, CA, 92121-2133, US

(73) Assignee: Burnham Institute for Medical Research, La Jolla, CA (US)

(21) Appl. No.: 12/424,354

(22) Filed: Apr. 15, 2009

Related U.S. Application Data
(60) Provisional application No. 61/057,121, filed on May 29, 2008.
(60) Provisional application No. 61/045,192, filed on Apr. 15, 2008.

(51) Int. Cl. A61K 31/426; C07D 277/34; A61P 35/00; C12Q 1/68; G01N 33/68; C12N 5/00; C12Q 1/18

(52) U.S. Cl. 514/369; 548/183; 435/6; 436/86; 435/377; 435/32

(57) ABSTRACT

A compound having the structure A is described as well as the use of such compounds to inhibit at least one BCL-2 protein family member.

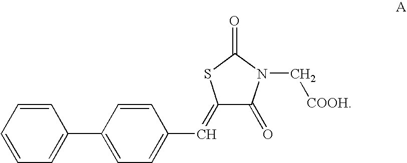

### CROSS-REFERENCE TO RELATED APPLICATIONS

This application claims priority under 35 U.S.C. §1 19(e) to U.S. Patent Application Ser. No. 61/057,121 filed May 29, 2008, and U.S. Patent Application Ser. No. 61/045,192 filed Apr. 15, 2008, each of which is hereby incorporated by reference in its entirety.

### BACKGROUND

1. Field of the Disclosure

The disclosure relates generally to a heterocyclic compound used for treating a variety of disorders, diseases and pathologic conditions, and more specifically, for treating cancer or autoimmune diseases.

2. Background Information

The apoptotic cascade in cells is known to lead to cell death. When anti-apoptotic proteins, such as BCL-2 family proteins, are overproduced by the cells, uncontrollable cell growth may ensue, potentially leading to the development of various serious diseases, disorders, and pathologies, particularly cancer.

Therefore, a need exists to inhibit anti-apoptotic proteins, such as the BCL-2 family proteins. Various potential BCL-2 antagonists have been previously identified. However, none of these compounds inhibits all six proteins in the BCL-2 family, i.e., all of the following proteins: BCL-X_{L}, BCL-2, BCL-W, BCL-B, BFL-1, and MCL-1. For example, none of the previously identified synthetic BCL-2 antagonists was effective at inhibiting the protein BFL-1. Therefore, the efficiency of such antagonists is not as high as desired. In addition, the existing antagonists are characterized by other drawbacks, such as insufficiency or safety issues.

In view of the above drawbacks and deficiencies of existing BCL-2 inhibitors, new antagonists of anti-apoptotic proteins, such as BCL-2 family proteins, are desired. It is desirable that such new antagonists be safer and more effective than the existing compounds.

### SUMMARY

According to one embodiment of the disclosure, there is provided a compound having the structure A, (Z)-2-(5-(biphenyl-4-ylmethylene)-2,4-dioxothiazolidin-3-yl)acetic acid, or pharmaceutically acceptable salts, hydrates, N-oxides, or solvates thereof:

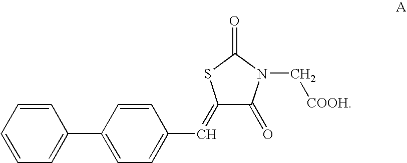

According to another embodiment of the disclosure, a method for treating cancer or autoimmune diseases is provided, comprising administering to a subject in need thereof a therapeutically effective amount of the compound having the structure A, or pharmaceutically acceptable salts, hydrates, N-oxides, or solvates thereof.

### BRIEF DESCRIPTION OF FIGURES

FIG. 1 demonstrates a predicted binding mode of compound A of the disclosure to a protein of the BCL-2 family.

FIG. 2 is a graphic representation on cell viability data.

FIG. 3 is a graphic representation of effects of compound A of the disclosure on shrinkage of B6Bcl2 spleen.

FIG. 4 is a graphic representation of effectiveness of compound A of the disclosure depending on the route of administration thereof.

### DETAILED DESCRIPTION

The following terms, definitions and abbreviations apply.

The term “patient” refers to organisms to be treated by the methods of the disclosure. Such organisms include, but are not limited to, humans. In the context of the disclosure, the term “subject” generally refers to an individual who will receive or who has received treatment described below (e.g., administration of the compounds of the disclosure, and optionally one or more additional therapeutic agents).

The term “BCL-2 family of proteins” refers to the family of proteins that currently includes at least the following six proteins: BCL-X_{L}, BCL-2, BCL-W, BCL-B, BFL-1, and MCL-1.

According to one embodiment of the disclosure, a compound having the structure A (having the chemical name (Z)-2-(5-(biphenyl-4-ylmethylene)-2,4-dioxothiazolidin-3-yl)acetic acid), or pharmaceutically acceptable salts, hydrates, N-oxides, or solvates thereof, are provided for treatment of various diseases, disorders, and pathologies:

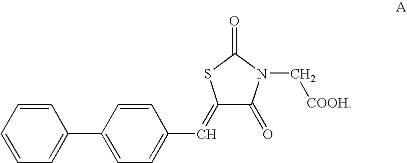

The compound of the disclosure includes any racemic, optically-active, polymorphic, or stereoisomeric form of compound A, or mixtures thereof, which possess the useful properties described herein. If desired, optically active forms can be prepared using commonly known techniques, e.g., by resolution of the racemic form by recrystallization techniques, by synthesis from optically-active starting materials, by chiral synthesis, or by chromatographic separation using a chiral stationary phase.

In one embodiment, a method is provided for inhibition of an anti-apoptotic family of proteins BCL-2. The method includes contacting a BCL-2 protein with compound A, under conditions that are favorable for contacting a BCL-2 protein and a compound of the disclosure. While not wanting to be bound to a particular mechanism, compound A is believed to be capable of inhibiting six proteins of the BCL-2 family, e.g., is capable of inhibiting all of such proteins as BCL-X_{L}, BCL-2, BCL-W, BCL-B, BFL-1, and MCL-1.

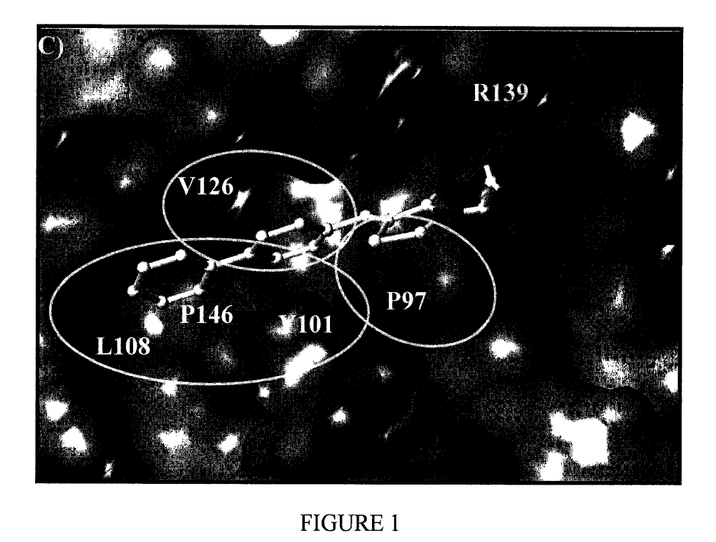

Predicted binding mode of compound A of the disclosure to a BCL-2 protein is illustrated by FIG. 1 supporting the conclusion that binding had occurred and indicating the site of binding in BCL-X_{L} protein.

The inhibition was also evaluated by measuring dissociation constant (K_{d}) values for compound A in comparison with some related compounds 1, 2, and 3. Such inhibition data are shown in Table 1. Stability data for compound A and compounds 1, 2, and 3 are also provided in Table 1 for reference.

TABLE 1
Selected Properties of Compound A

| Com- | | | | |
|---|---|---|---|---|
| pound | A | 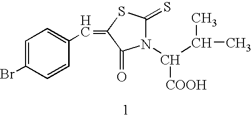 | 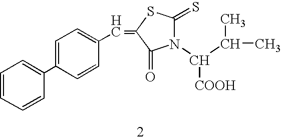 | 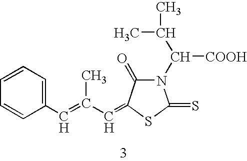 |
| K_{d}, μM | 14.0 | 1.1 | 0.16 | 11.8 |
| Plasma | 58 | 56 | 59 | 51 |
| Stabil- | | | | |
| ity, % | | | | |
| (45 | | | | |
| min- | | | | |
| utes) | | | | |
| Micro- | 72 | 54 | 22 | 75 |
| somal | | | | |
| Stabil- | | | | |
| ity, % | | | | |
| (45 | | | | |
| min- | | | | |
| utes) | | | | |

As can be seen from the data presented in Table 1, compound A of the disclosure possesses the inhibition activity that is better that that of any of the related compounds 1, 2, and 3, and is vastly superior to that of either compound 1 or compound 2. Stability data provided in Table 1 also demonstrates that compound A has stability that is at least comparable to that of compounds 1, 2, and 3, or even has better stability.

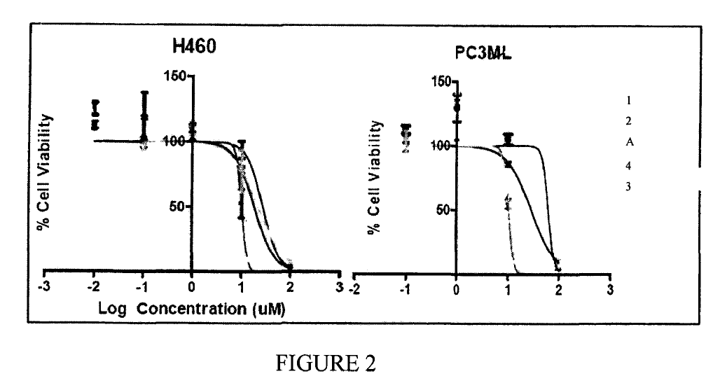

The inhibition information for cells H460 and PC3ML is also shown by FIG. 2. As can be clearly seen, compound A has the largest influence on the cell viability, in comparison with other related compounds 1-4, both for the viability data of H460 and PC3ML. The structures of compounds 1-3 are shown in Table 1, above, and the structure of compound 4 is as follows:

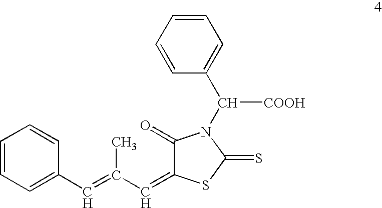

According to other embodiments, a method is provided for treating a disease or disorder. The method can include administering to a subject in need of such treatment, an effective amount of any above-described compound, or pharmaceutically acceptable salts, hydrates, or solvates thereof. Non-limiting examples of the diseases or disorders that can be treated are cancer and autoimmune diseases.

According to another embodiment, a method is provided for treating cancer. The method comprises administering to a subject in need thereof a therapeutically effective amount of the above-described compound A, or a pharmaceutically acceptable salt, hydrate, N-oxide, or solvate thereof. Compound A may be used for treating any type of cancer. In some aspects, the kinds of cancer that may be treated include lung cancer, breast cancer, prostate cancer, as well as a variety of lymphomas.

Compound A was tested in vivo in the B6BCL-2 transgenic mouse, and shown in vivo activity that was equal to, or better than, known compounds gossypol and apogossypol. In the same model, another known compound apogossypolone was not effective. Gossypol is described, e.g., in U.S. Pat. No. 7,186,708. Apogossypol is described, e.g., in Meyers A. I.; Willemsen J. J., Tetrahedron Letters, vol. 37, No. 6, February, 51996, pp. 791-792. The potency of the compounds in terms of in vivo efficacy in this mouse model was in the following order: compound A>apogossypol=gossypol.

According to another embodiment, compound A can be used for the manufacture of a medicament for the treatment of a pathological condition or symptom in a mammal, such as a human. The medicament can be directed to the treatment of cancer, within the limitations described above.

According to another embodiment, pharmaceutical compositions are provided, the pharmaceutical compositions comprising compound A, or pharmaceutically acceptable salts, hydrates, or solvates thereof, and a pharmaceutically acceptable diluent or carrier. The pharmaceutical compositions can be used to treat cancer. The pharmaceutical compositions can further optionally include one or more additional therapeutic anti-cancer agents, including, but not limited to, such agents as (1) alkaloids, including, microtubule inhibitors (e.g., Vincristine, Vinblastine, and Vindesine, etc.), microtubule stabilizers (e.g., Paclitaxel [Taxol], and Docetaxel, Taxotere, etc.), and chromatin function inhibitors, including, topoisomerase inhibitors, such as, epipodophyllotoxins (e.g., Etoposide [VP-16], and Teniposide [VM-26], etc.), and agents that target topoisomerase I (e.g., Camptothecin and Isirinotecan [CPT-11], etc.); (2) covalent DNA-binding agents [alkylating agents], including, nitrogen mustards (e.g., Mechlorethamine, Chlorambucil, Cyclophosphamide, Ifosphamide, and Busulfan [Myleran], etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); (3) noncovalent DNA-binding agents [antitumor antibiotics], including, nucleic acid inhibitors (e.g., Dactinomycin [Actinomycin D], etc.), anthracyclines (e.g., Daunorubicin [Daunomycin, and Cerubidine], Doxorubicin [Adriamycin], and Idarubicin [Idamycin], etc.), anthracenediones (e.g., anthracycline analogues, such as, [Mitoxantrone], etc.), bleomycins (Blenoxane), etc., and plicamycin (Mithramycin), etc.; (4) antimetabolites, including, antifolates (e.g., Methotrexate, Folex, and Mexate, etc.), purine antimetabolites (e.g., 6-Mercaptopurine [6-MP, Purinethol], 6-Thioguanine [6-TG], Azathioprine, Acyclovir, Ganciclovir, Chlorodeoxyadenosine, 2-Chlorodeoxyadenosine [CdA], and 2′-Deoxycoformycin [Pentostatin], etc.), pyrimidine antagonists (e.g., fluoropyrimidines [e.g., 5-fluorouracil (Adrucil), 5-fluorodeoxyuridine (FdUrd) (Floxuridine)] etc.), and cytosine arabinosides (e.g., Cytosar [ara-C] and Fludarabine, etc.); (5) enzymes, including, L-asparaginase, and hydroxyurea, etc.; (6) hormones, including, glucocorticoids, such as, antiestrogens (e.g., Tamoxifen, etc.), nonsteroidal antiandrogens (e.g., Flutamide, etc.), and aromatase inhibitors (e.g., anastrozole [Arimidex], etc.); (7) platinum compounds (e.g., Cisplatin and Carboplatin, etc.); (8) monoclonal antibodies conjugated with anticancer drugs, toxins, and/or radionuclides, etc.; (9) biological response modifiers (e.g., interferons [e.g., IFN-.alpha., etc.] and interleukins [e.g., IL-2, etc.], etc.); (10) adoptive immunotherapy; (11) hematopoietic growth factors; (12) agents that induce tumor cell differentiation (e.g., all-trans-retinoic acid, etc.); (13) gene therapy agents; 14) antisense therapy agents; (15) tumor vaccines; (16) agents directed against tumor metastases (e.g., Batimistat, etc.); (17) inhibitors of angiogenesis, and (18) selective serotonin reuptake inhibitors (SSRI's).

Representative, but non-limiting examples of suitable SSRIs that may be used include sertraline (e.g., sertraline hydrochloride, marketed under the trademark “Zoloft®” by Pfizer, Inc.) or sertraline metabolite, fluvoxamine (e.g., fluvoxamine melate, marketed under the trademark “Luvox®” by Solvay Pharmaceuticals, Inc.), paroxetine (e.g., paroxetine hydrochloride, marketed under the trademark “Paxil®” by SmithKline Beecham Pharmaceuticals, Inc.), fluoxetine (e.g., fluoxetine hydrochloride, marketed under the trademarks “Prozac®” or “Sarafem®” by Eli Lilly and Company) and citalopram (e.g., citalopram hydrobromide, marketed under the trademark “Celexa®” by Forest Laboratories, Parke-Davis, Inc.), and metabolites thereof. Additional examples include venlafaxine (e.g., venlafaxine hydrochloride marketed under the trademark “Effexor®” by Wyeth-Ayerst Laboratories), mirtazapine (e.g., marketed under the trademark “Remeron®” by Organon, Inc.), buspirone (e.g., buspirone hydrochloride marketed under the trademark “Buspar®” by Bristol-Myers Squibb), trazodone (e.g., trazodone hydrochloride marketed under the trademark “Desyrel®” by Bristol-Myers Squibb and Apothecon), nefazadone (e.g., nefazodone hydrochloride marketed under the trademark “Serzon®” by Bristol-Myers Squibb), clomipramine (e.g., clomipramine hydrochloride marketed under the trademark “Anafranil®” by Novopharm, LTD, Ciba, and Taro Pharmaceuticals), imipramine (e.g., imipramine hydrochloride marketed under the trademark “Tofranil®” by Glaxo-Welcome, Inc.), nortriptyline (e.g., Nortriptyline hydrochloride marketed under the trademark “Nortrinel®” by Lundbeck), mianserine (e.g., marketed under the trademark “Tolvon®” by Organon, Inc.), duloxetine (e.g., duloxetine hydrochloride marketed by Eli Lilly and Company), dapoxetine (e.g., dapoxetine hydrochloride marketed by ALZA Corporation), litoxetine (e.g., litoxetine hydrochloride marketed by Synthelabo Recherche (L.E.R.S.), Bagneux, France.), femoxetine, lofepramine (e.g., marketed under the trademark “Gamonil®” by MERCK & Co., Inc.), tomoxetine (e.g., marketed by Eli Lilly and Company). The disclosure encompasses SSRIs that are currently used, or those later discovered or formulated. SSRIs, including those listed above, may be administered orally in an amount between about 2 mg and about 2,500 mg daily.

In the broad sense, any cancer or tumor (e.g. hematologic and solid tumors) may be treated according to embodiments of the disclosure. Exemplary cancers that may be treated according to embodiments of the disclosure include, but are not limited to, head and neck cancer, brain cancer (e.g. glioblastoma multifoma) breast cancer, colorectal cancer, esophageal cancer, gastric cancer, hepatic cancer, bladder cancer, cervical cancer, endometrial cancer, lung cancer (non-small cell), ovarian cancer and other gynological cancers (e.g. tumors of the uterus and cervix), pancreatic cancer, prostate cancer, renal cancer, choriocarcinoma (lung cancer), skin cancer (e.g. melanoma, basal cell carcinoma), hairy cell leukemia, chronic lymphotic leukemia, acute lymphocytic leukemia (breast & bladder), acute myelogenous leukemia, meningeal leukemia, chronic myelogenous leukemia, and erythroleukemia. More commonly, the cancers treated include leukemia and B-cell cancers (e.g. lymphoma, multiple myeloma, and MDS.

The biological activity of compounds provided herein can be evaluated by in vitro and in vivo assays and procedures known in the art, including for example those described in Alley, M. C., et. al. Feasibility of Drug Screening with Panels of Human Tumor Cell Lines Using a Microculture Tetrazolium Assay. Cancer Research 48: 589-601, 1988; Grever, M. R., et. al. The National Cancer Institute: Cancer Drug Discovery and Development Program. Seminars in Oncology, Vol. 19, No. 6, pp 622-638, 1992; Boyd, M. R., and Paull, K. D. Some Practical Considerations and Applications of the National Cancer Institute In Vitro Anticancer Drug Discovery Screen. Drug Development Research 34: 91-109, 1995; Shoemaker, R. H. The NCI60 Human Tumour Cell line Anticancer Drug Screen. Nature Reviews, 6: 813-823, 2006, each of which is incorporated by reference in its entirety.

Non-limiting examples of autoimmune diseases that can be treated using the above-described compound A and methods of the disclosure include rheumatoid arthritis, psoriatic arthritis, juvenile idiopathic arthritis, multiple sclerosis, systemic lupus erythematosus, myasthenia gravis, juvenile onset diabetes, glomerulonephritis, autoimmune thyroiditis, Behcet's disease, Crohn's disease, ulcerative colitis, bullous pemphigoid, sarcoidosis, psoriasis, ichthyosis, Graves ophthalmopathy, psoriasis, psoriasis inflammatory bowel disease, and asthma.

In some cases, it may be appropriate to administer compound A of the disclosure as a salt. Examples of pharmaceutically acceptable salts include organic acid addition salts formed with acids which form a physiological acceptable anion, for example, tosylate, methanesulfonate, acetate, citrate, malonate, tartarate, succinate, benzoate, ascorbate, ketoglutarate, and glycerophosphate. Suitable inorganic salts may also be formed, including hydrochloride, sulfate, nitrate, bicarbonate, and carbonate salts. Pharmaceutically acceptable salts may be obtained using standard procedures well known in the art, for example by reacting a compound A with a suitable base affording a physiologically acceptable anion. Alkali metal (for example, sodium, potassium or lithium) or alkaline earth metal (for example calcium) salts of carboxylic acids can also be made.

Any tablets, troches, pills, capsules, and the like, which incorporate compound A, may also contain binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When there is a unit dosage form of compound A, it may contain, in addition to materials of the above type, a liquid carrier, such as a vegetable oil or a polyethylene glycol. Various other materials may be present as coatings or to otherwise modify the physical form of a solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form should be pharmaceutically acceptable and substantially non-toxic in the amounts employed. In addition, compound A may be incorporated into sustained-release preparations and devices.

Compound A may also be administered intravenously or intraperitoneally by infusion or injection. Solutions of compound A may be prepared in water, optionally mixed with a nontoxic surfactant. Dispersions may also be prepared in glycerol, liquid polyethylene glycols, triacetin, and mixtures thereof and in oils. Under ordinary conditions of storage and use, these preparations may contain a preservative to prevent the growth of microorganisms.

Sterile injectable solutions can be prepared by incorporating compound A of in the sufficient therapeutic amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compound A may be applied in pure form, i.e., when it is a liquid. However, it will generally be desirable to administer it to the skin as compositions or formulations, in combination with a dermatologically acceptable carrier, which may be a solid or a liquid. Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants and additional antimicrobial agents can be added to optimize the properties for a given use.

The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user, as known to those having ordinary skill in the art.

### EXAMPLES

Some aspects of the disclosure can be further illustrated by the following non-limiting examples.

### Example 1

### Protein Expression and Purification

Recombinant full length BCL-XL was produced from a pET-19b (Novagen) plasmid construct containing the entire nucleotide sequence for BID fused to an N-terminal poly-His tag. Unlabeled protein was expressed in E. coli BL2 1 in LB media at 37° C., with an induction period of 3-4 hours with 1 mM IPTG. ¹⁵N-labeled protein was similarly produced, with growth occurring in M9 media supplemented with 0.5 g/L ¹⁵NH₄Cl. Following cell lysis, soluble protein was purified over a Hi-Trap chelating column (Amersham, Pharmacia), followed by ion-exchange purification with a MonoQ (Amersham, Pharmacia) column. Final BID samples were dialyzed into a buffer appropriate for the subsequent experiments.

### Example 2

### Molecular Modeling

Molecular modeling studies were conducted on several R12000 SGI Octane workstations with the software package Sybyl version 6.9 (TRIPOS). The docked structures of the compounds were initially obtained by Gold. Molecular models of compounds were energy-minimized with MAXIMN2 (Sybyl). For each molecule, 20 solutions were generated and ranked according to Goldscore. The solutions were finally ranked by visual inspection of the linked compounds in the deep hydrophobic groove on the surface of BCL-xL. Surface representations were generated by MOLCAD.

### Example 3

### NMR Spectroscopy

For all NMR experiments, BCL-xL was exchanged into 50 mM phosphate buffer at pH 7.5 and measurements were performed at 30° C. 2D [¹⁵N,¹H]-HSQC spectra for BCL-xL were measured with 0.5 mM samples of ¹⁵N-labeled protein. All experiments were performed with a 600 MHz Bruker Avance spectrometer, both equipped with either a TXI probe or a TCI cryoprobe. In all experiments, dephasing of residual water signals was obtained with a WATERGATE sequence. In order to test the ability of test compounds to bind to Bcl-xL, a 25 μM sample of the protein was prepared and 1D ¹H NMR spectra were collected in absence and presence of test compounds. By observing the aliphatic region of the spectra, binding can be readily detected in these simple experiments due to chemical shift changes in active site methyl groups of Ile, Leu, Thr, Val or Ala (region between 0.8 and 0.3 ppm).

### Example 4

### Synthetic Procedures

Compound A was synthesized according to the flowing synthetic scheme:

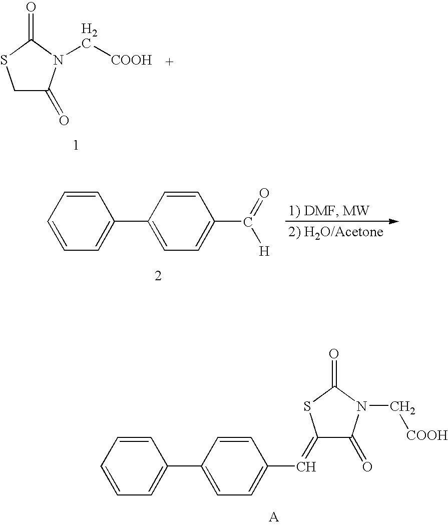

2-(2,4-dioxothiazolidin-3-yl)acetic acid (1) was added to a solution of the biphenyl-4-carbaldehyde (2) (1:1 mmol ratio) in dimethylformamide (1 ml) and the mixture was stirred until it became homogenous. The mixture is then placed in the microwave, where it underwent four cycles of 10-min heating (140° C., 1,000 W) and 5 min of cooling at 25° C. Water was then added to the solution where precipitate was formed. The precipitate was then collected via filtration, recrystallized from acetone/water, and dried to yield the desired compound A.

Yield 58%; white solid; ¹H NMR (600 MHz, DMSO-d6): δ 4.3 (s, 2H); 7.42 (m, ¹H); 7.5 (d, 2H, J=7.2 Hz); 7.76 (m, 4H); 7.87 (d, 2H, J=7.8 Hz); 8.02 (s, 1H). Calcd for C₁₈H₁₃NO₄S: C, 63.71; H, 3.86; N, 4.13; S, 9.45; Found: C, 62.54; H, 4.31; N, 4.12; S, 8.47.

### Example 5

### Effectiveness of Compound A In Vivo

Compound A was given to B6Bcl2 mice at a daily dose of 12 mmol/kg for 3 days through oral gavage. As a negative control, rhodanine acetic acid (which does not bind to Bcl-xL) was given at a daily dose of 12 mmol/kg for 3 days in the same manner. Both compound A and the negative control were preliminarily dissolved in PBS. After 3 days, the spleens of the animals were removed and weighed.

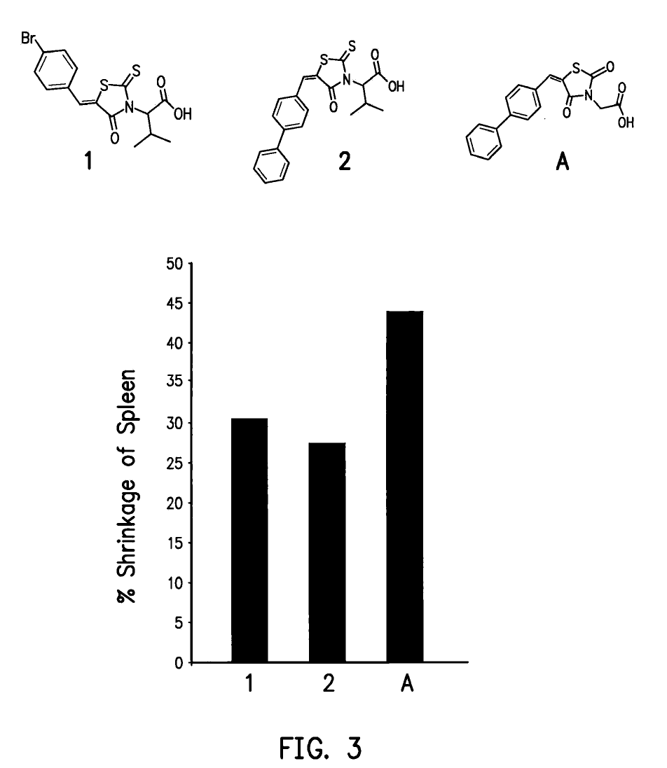

In parallel experiments, compound A was also administered intraperitoneally at 60 mmol/kg, as were some related compounds, such as compounds 1 and 2 shown in Table 1, above. In these experiments, after 24 hours, the spleens of the animals were removed and weighed. Compound A showed efficacy that was superior to that of either compound 1 or compound 2, inducing the degree shrinkage of spleen that was about 40% higher than the shrinkage induced by compounds 1 or 2, as can be seen from FIG. 3.

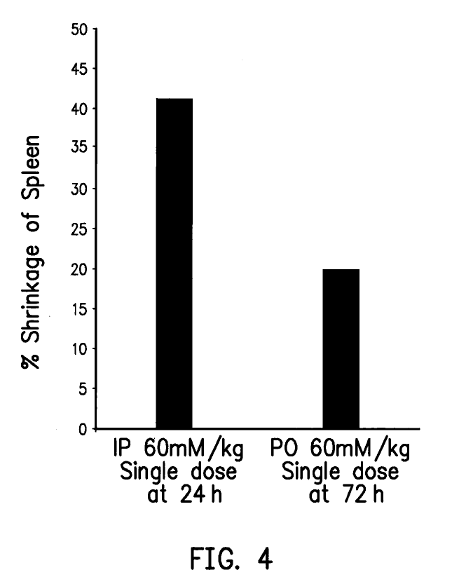

The results of efficacy of compound A administered intraperitoneally were also compared with the results obtained when compound A was administered orally. The results indicate that compound A induced shrinkage of spleen in experiments eploying either type of adminustration. Accordingly, compound A can be administered in both ways, orally or intraperitoneally. However, intraperitoneal injection induced about 100% higher degree of shrinkage than oral dosing, as demonstrated by FIG. 4. It was also shown that compound A can be administered safely. There was no weight loss or signs of toxicity via physical exam regardless of the selected route of administration.

The effectiveness of compound A was also evaluated, in comparison with compound 1 shown in Table 1, by determining mean IC₅₀ values, which were measured for three independent experiments (each in triplicate) for compounds 1 and A. All points were normalized to control as a percentage of cell viability and statistics were completed with Graphpad Prism software. The results presented in Table 2, which also provide standard deviation data, demonstrate superior effectiveness of compound A.

TABLE 2
Inhibition Data for Compound A

| | | IC₅₀, μM for | IC₅₀, μM for |
|---|---|---|---|
| No. | Cell Line | Compound 1 | Compound A |
| 1 | A549L | 12.2 ± 5.9 | 0.8 ± 0.3 |
| 2 | H460 | 3.0 ± 2.4 | 0.5 ± 0.3 |
| 3 | RS11846S | 6.5 ± 10.0 | 0.4 ± 1.4 |
| 4 | PC3ML | 60.9 ± 1.6 | 0.7 ± 0.6 |

Although the invention has been described with reference to the above examples, it will be understood that modifications and variations are encompassed within the spirit and scope of the invention. Accordingly, the invention is limited only by the following claims.

What is claimed is:

1. A compound having the formula A, or a pharmaceutically acceptable salt, hydrate, N-oxide, or solvate thereof:

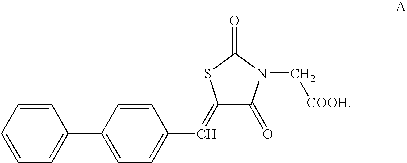

2. (Z)-2-(5-(biphenyl-4-ylmethylene)-2,4-dioxothiazolidin-3-yl)acetic acid, or a pharmaceutically acceptable salt, hydrate, N-oxide, or solvate thereof.

3. A method for treating a disease or a disorder, comprising administering to a subject in need thereof a therapeutically effective amount of the compound of claim 1 or 2, or a pharmaceutically acceptable salt, hydrate, N-oxide, or solvate thereof, thereby treating the disease or the disorder.

4. The method of claim 3, wherein the disease or the disorder is cancer.

5. The method of claim 4, wherein cancer is selected from the group consisting of lung cancer, breast cancer, prostate cancer, and lymphomas.

6. The method of claim 5, wherein the treatment includes inhibition of activity of at least one BCL-2 family protein.

7. The method of claim 3, comprising administering the compound in combination with an anti-cancer agent.

8. A method of treating cancer or an autoimmune disease in a subject having at least one elevated BCL-2 family protein expression level comprising administering to the subject a therapeutically effective amount of a compound having the structure A, or a pharmaceutically acceptable salt, hydrate, N-oxide, or solvate thereof:

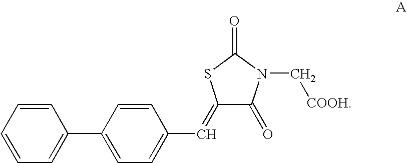

9. The method of claim 8, further comprising determining whether the subject is responsive to a therapy that utilizes the compound having the structure A, or a pharmaceutically acceptable salt, hydrate, N-oxide, or solvate thereof, comprising determining the level of at least one of the BCL-2 family protein in the subject and comparing to a normal control sample, wherein an elevated level is indicative of a subject responsive to the therapy that utilizes compound having the structure A, or a pharmaceutically acceptable salt, hydrate, N-oxide, or solvate thereof.

10. A method of determining whether a subject is responsive to a therapy that utilizes a compound having the structure A, or a pharmaceutically acceptable salt, hydrate, N-oxide, or solvate thereof: 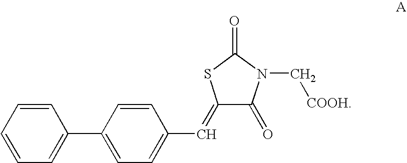
comprising determining the level of at least one of the BCL-2 family protein in the subject and comparing to a normal control sample, wherein an elevated level is indicative of a subject responsive to the therapy that utilizes the compound having the structure A, or a pharmaceutically acceptable salt, hydrate, N-oxide, or solvate thereof.

11. The method of claim 9 or 10, wherein the determination is made based on a sample from the subject.

12. The method of claim 11, wherein the sample is a biological fluid or tumor sample.

13. The method of claim 9 or 10, wherein the BCL-2 family polynucleotide or polypeptide is selected from BCL-2, BCL-XL, BCL-W, MCL-1, and BCL-A1.

14. A method of inducing apoptosis in a cell having a level of at least one of the BCL-2 family protein member greater than levels in a control cell, comprising administering to the cell an effective amount of a compound having the structure A, or a pharmaceutically acceptable salt, hydrate, N-oxide, or solvate thereof: 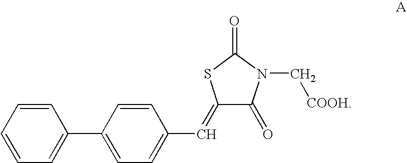
to reduce the level of Bcl-2 family protein(s) and induce apoptosis in the cell.

15. The method of claim 14, wherein the cell is a cancer cell.

16. The method of claim 15, wherein cancer is selected from the group consisting of lung cancer, breast cancer, prostate cancer, and lymphomas.

17. The method of claim 14, wherein the cell is a cell of the immune system.

18. A method of determining the effectiveness of a therapeutic regimen including administration of a compound having the structure A, or a pharmaceutically acceptable salt, hydrate, N-oxide, or solvate thereof: 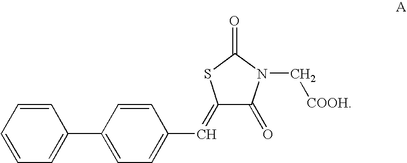
in a subject comprising comparing the level of a BCL-2 family protein in a cell of the subject prior to and during treatment with the compound having the structure A, or a pharmaceutically acceptable salt, hydrate, N-oxide, or solvate thereof, wherein a decreased level of BCL-2 family protein is indicative of effectiveness of the therapy that utilizes the compound having the structure A, or a pharmaceutically acceptable salt, hydrate, N-oxide, or solvate thereof.

19. The method of claim 18, wherein the subject has cancer.

20. The method of claim 19, wherein cancer is selected from the group consisting of lung cancer, breast cancer, prostate cancer, and lymphomas.

21. The method of claim 18, wherein the subject has an autoimmune disorder.
